(19) United States
(12) Patent Application Publication
(10) Pub. No.: US 2011/0014683 A1
(43) Pub. Date: Jan. 20, 2011

(54) System and Method for Growing Photosynthetic Cells

(75) Inventors: Willem F. J. Vermaas, Tempe, AZ (US); Bruce E. Rittmann, Tempe, AZ (US)

Correspondence Address: FULBRIGHT & JAWORSKI L.L.P., 600 CONGRESS AVE., SUITE 2400, AUSTIN, TX, 78701, US

(73) Assignee: ARIZONA BOARD OF REGENTS FOR AND ON BEHALF OF ARIZONA STATE UNIVERSITY, Scottsdale, AZ (US)

(21) Appl. No.: 12/446,405

(22) PCT Filed: Oct. 19, 2007
(86) PCT No.: PCT/US07/81994

§ 371 Date: Sep. 23, 2010

Related U.S. Application Data
(60) Provisional application No. 60/862,366, filed on Oct. 20, 2006.

(51) Int. Cl. C12N 1/20; C12M 1/04

(52) U.S. Cl. 435/252.1; 435/292.1

(57) ABSTRACT

Disclosed are a system and method for growing photosynthetic cells in conduit. The system and method supply light, CO2 and nutrients to the cells. The system and method also dampen thermal variations in the conduit.

### CROSS-REFERENCE TO RELATED APPLICATIONS

This application claims the priority of U.S. Provisional Patent Application Ser. No. 60/862,366, filed Oct. 20, 2006, entitled “System and Method for Growing Cells”, the entire disclosure of which is specifically incorporated herein by reference.

### BACKGROUND OF THE INVENTION

A. Field of the Invention

Embodiments of the present invention relate generally to a system and method for growing photosynthetic cells under controlled conditions. In particular, embodiments of the present invention concern the use of photosynthetic microorganisms to produce products such as biofuels.

B. Description of Related Art

Two challenges facing the world today include the ongoing pollution of the environment with carbon dioxide, which contributes to global warming and the increasing consumption of the world's natural energy resources such as fossil fuels. A problematic cycle exists where the increase in fossil fuel consumption correlates with an increase in carbon dioxide air pollution.

For instance, it has been estimated that the United States produces 1.7 billion tons of carbon dioxide annually from the combustion of fossil fuels (see U.S. Publication No. 2002/0072109). Global production of carbon dioxide from fossil fuel consumption is much greater and estimated to be between 7-8 billion tons/year (Marland et al. 2006). An increase in carbon dioxide air pollution can lead to an increase in global warming, which in turn can increase the frequency and intensity of extreme weather events, such as floods, droughts, heat waves, hurricanes, and tornados. Other consequences of global warming can include changes in agricultural yields, species extinctions, and increases in the ranges of disease vectors.

Methods for carbon dioxide remediation have been suggested. For instance, U.S. Publication No. 2002/0072109 discloses an on-site biological sequestration system that can decrease the concentration of carbon-containing compounds in the emissions of fossil generation units. The system uses photosynthetic microbes, such as algae and cyanobacteria, which are attached to a growth surface arranged in a containment chamber that is lit by sunlight. The cyanobacteria or algae uptake the carbon dioxide produced by the fossil generation units.

As for the second challenge, increasing global energy demand places a higher demand on the non-renewable fossil fuel energy supplies. Alternative sources for energy are being developed. For instance, agricultural products such as corn, soybeans, flaxseed, rapeseed, sugar cane, and palm oil are currently being grown for use in biofuel production. Biodegradable by-products from industries such as the agriculture, housing, and forestry industries can also be used to produce bioenergy. For example, straw, timber, manure, rice, husks, sewage, biodegradable waste and food leftovers can be converted into biogas through anaerobic digestion.

Methods for using living organisms to produce ethanol have also been attempted. For instance, U.S. Pat. No. 4,242,455 to Muller et al. describes a continuous process in which an aqueous slurry of carbohydrate polymer particles, such as starch granules and/or cellulose chips, and fibers, are acidified with a strong inorganic acid to form a fermentable sugar. The fermentable sugar is then fermented to ethanol with at least two strains of Saccharomyces. U.S. Pat. No. 4,350,765 to Chibata et al. describes a method of producing ethanol in a high concentration by using an immobilized Saccharomyces or Zymomonas and a nutrient culture broth containing a fermentative sugar. U.S. Pat. No. 4,413,058 to Arcuri et al. describes a strain of Zymomonas mobilis, which is used to produce ethanol by placing the microorganism in a continuous reactor column and passing a stream of aqueous sugar through said column.

PCT Application WO/88/09379 to Hartley et al. describes the use of facultative anaerobic thermophilic bacterial strains that produce ethanol by fermenting a wide range of sugars, including cellobiose and pentoses. These bacterial strains contain a mutation in lactate dehydrogenase. As a result, these strains, which would normally produce lactate under anaerobic conditions, produce ethanol instead.

U.S. Publication 2002/0042111 discloses a genetically modified cyanobacterium that can be used to produce ethanol. The cyanobacterium includes a construct comprising DNA fragments encoding pyruvate decarboxylase (pdc) and alcohol dehydrogenase (adh) enzymes obtained from the Zymomonas mobilis plasmid pLOI295.

### SUMMARY

Embodiments of the present disclosure overcome deficiencies in the art by providing a versatile and controllable system and method for growing photosynthetic cells. The systems and methods allow independent control of the factors that determine the physiological characteristics of the photosynthetic microorganisms and their production of valuable products. Embodiments also minimize consumption of energy and water during the system's operation.

In certain embodiments, the system comprises a conduit with an outer surface, an inner surface, an inner volume, a length, and at least a portion that permits sunlight to pass into the inner volume during use. At least a portion of the conduit can be exposed to sunlight during the day, and a thermal dampening system can be in operable relationship to the conduit. In non-limiting examples, a CO₂ supply system is configured to supply CO₂ to the inner volume during use; a nutrient supply system is configured to supply one or more nutrients (for example, nitrogen and phosphorus) to the inner volume during use; and a separation system to remove cells from the conduit during use and to return cells and filtered water back to the inner volume in a controlled manner. In certain embodiments, the nutrient may be a component of nitrate or another fixed nitrogen compound.

In non-limiting examples, the thermal dampening system may comprise a pond between about two feet deep and ten feet deep, preferably between four and six feet deep, and most preferably about five feet deep. The pond may be between 50 meters square and 200 meters square, preferably between 100 meters square and 150 meters square, and most preferably around 130 meters square. The pond may be formed by earthen embankments in a near-level land area. The conduit may be submerged more or less than three feet below the surface of the pond and the pond may be partially or completely shaded. Portions of the conduit may also be underground or shielded from outside light in some other manner. A shading system may comprise a retractable tarp drawn by cables or chain drives or a retractable swimming pool cover. The pond may also be divided into segments so that different operating conditions can be maintained in different segments. Moreover, two or more ponds containing conduit may be operated in parallel to scale up to larger production rates of photosynthetic cells. The thermal dampening system may also comprise catwalks over the pond or other fluid reservoir. The catwalks may run longitudinally along the conduit and across the conduit at approximately six to eight foot centers and may be supported from the bottom of the reservoir. The catwalks may be used for maintenance and cleaning of the conduit. In non-limiting examples, the system may comprise cleaning devices for cleaning the conduit, such as soft balls dragged through the conduit to clean the inside of the conduit. In certain embodiments, brushes, vacuums, or hydroblasters may be used to clean the outside of the conduit.

Certain embodiments may comprise a fluid control system configured to: remove a substantially solids-free permeate from the conduit; recycle a portion of the substantially solids-free permeate back to the conduit; remove a concentrated-solids retentate from the system; and recycle a portion of the substantially solids-free permeate back to the conduit.

The photosynthetic cells may be cyanobacteria according to the U.S. Provisional Patent Application Ser. No. 60/853,285, entitled “Modified Cyanobacteria”, filed on or about Oct. 20, 2006 and PCT Application No. ______, entitled “Modified Cyanobacteria”, filed on or about Oct. 20, 2007, by Willem F. J. Vermaas, incorporated herein by reference. In non-limiting examples, the cyanobacteria may be Synechocystis sp. PCC 6803 or Thermosynechococcus elongatus sp. BP-1.

Synechocystis sp. PCC 6803 is a unicellular organism that displays a unique combination of highly desirable molecular genetic, physiological, and morphological characteristics. For instance, this species is spontaneously transformable, incorporates foreign DNA into its genome by double-homologous recombination, grows under many different physiological conditions (e.g., photoauto/mixo/heterotrophically), and is relatively small (˜1.5 μm in diameter) (Van de Meene et al. 2006). Its entire genome has been sequenced (Kaneko et al. 1996), and a high percentage of open reading frames without homologues in other bacterial groups have been found (Fraser et al. 2000). Synechocystis sp. PCC6803 is available from the American Type Culture Collection, accession number ATCC 27184 (Rippka et al., 1979. J. Gen. Micro., 111:1-61).

Thermosynechococcus elongatus sp. BP-1 is a unicellular thermophilic cyanobacterium that inhabits hot springs and has an optimum growth temperature of approximately 55° C. (Nakamura et al. 2002). The entire genome of this bacterium has been sequenced. The genome includes a circular chromosome of 2,593,857 base pairs. A total of 2475 potential protein-encoding genes, one set of rRNA genes, 42 tRNA genes representing 42 tRNA species and 4 genes for small structural RNAs were predicted.

The portion of the conduit that permits sunlight to pass into the inner volume during use may be clear. In certain embodiments, the clear portion may be comprised of clear or translucent glass, polyvinyl chloride, polycarbonate, or polyethylene, and the conduit may comprise a tube with a circular cross-section.

In other embodiments, a method of growing cells comprises culturing cells in an inner volume of one or more conduits; supplying CO₂ and fixed nitrogen to the inner volume; exposing the CO₂ and fixed nitrogen to natural light; dampening any thermal variations in the conduits; and removing cells from the inner volume. In certain embodiments the cells are cyanobacteria, and dampening thermal variations comprises contacting an outer surface of the conduits with a fluid. In non-limiting examples the temperature of the fluid is controlled, and the flow rates through the conduits are between 2 and 20 cm/sec, more preferably between 4 and 10 cm/sec, and most preferably 5-10 cm/sec. The CO₂ may be supplied by a flue gas or combustion off gas, and the nutrients may be supplied by ground water, ammonia, nitrate, or another fixed nitrogen compound. In non-limiting examples, the cells are removed by a membrane and the conduits are submerged in a fluid reservoir.

In certain non-limiting methods, the CO₂, fixed nitrogen and temperature in the inner volume are maintained at amounts suitable for growing cyanobacteria or other photosynthetic microorganisms. For example, the CO₂ may be maintained at about 0.01% to 10%, more preferably between 0.02% and 7%, and most preferably between 0.03% to 5% in the inner volume of the conduit. The nitrogen may be maintained at approximately 0.1 to 15 mM (millimolar), preferably between 0.3 and 12 mM. The temperature may be maintained at approximately 3-80 degrees Celsius, preferably 10-60 degrees Celsius in the inner volume.

In non-limiting examples, the conduit may be between about 1 and 18 inches in diameter, more preferably between 4 and 8 inches in diameter, and most preferably about 5-7 inches in diameter. The conduit may be between 10 and 200 meters long, preferably between 50 and 150 meters long, and most preferably around 100 meters long.

The thermal dampening system may be configured to circulate a fluid in contact with the conduit during use and may comprise a fluid reservoir with a synthetic liner in which at least a portion of the conduit is submerged in the fluid reservoir. In other embodiments, the system may comprise a support configured to support the conduit. The supports may comprise pipe stands or corrugated sheets and may be spaced approximately 2 to 50 feet apart, more preferably between about 4 and 10 feet apart, and most preferably about 6 feet apart. In non-limiting examples, there are several rows of conduit connected to distribution headers at one or both ends of the conduit rows. In certain embodiments, the flow in approximately half of the conduit rows is in one direction and the flow in the remaining conduit rows is in the opposite direction.

In certain embodiments, the CO₂ supply system may comprise a pump and may be configured to inject combustion off or flue gas into the inner volume of the conduit during use.

In non-limiting examples, the pump used to circulate the fluid within the conduit may be an airlift pump, an axial flow pump, a centrifugal pump, a screw pump, or a positive displacement pump. It may provide a flow rate of approximately 500 to 5,000 L/min, more preferably between about 1,000 and 3,000 L/min and most preferably about 2,500 L/min. In non-limiting examples, the pump may provide flow at a total dynamic head of approximately 0.25 to 10 meters, more preferably between 0.5 and 5 meters, and most preferably about 1.0 meters.

In other examples, the system comprises a distribution trough or header in operable relationship with the conduit and the distribution trough or header is configured to receive CO₂ injection during use. The CO₂ may be provided from a number of different sources, including those that provide a combustion off-gas. The CO₂ supply system may also comprise an air blower that supplies the CO₂-containing gas. The air blower may have a flow rate of approximately 100 to 5,000 cubic meters per hour, more preferably between 500 and 2,500 and most preferably about 1,500 cubic meters per hour. In certain embodiments, the CO₂ system may have a scrubber (for example, an alkali scrubber) to remove contaminants (for example, SO₂). In a non-limiting example, a UV light or chemical filter may be used to sterilize air from the air blower. The CO₂ supply system may be configured to inject CO₂ directly into the distribution header or trough.

In certain embodiments, the nutrient-supply system may be configured to supply ground water to the inner volume of the conduit during use. In this example, a synergistic benefit is realized with the ground water providing nitrogen to the system and the system removing nitrogen from the groundwater. The nutrient supply system may also comprise a storage tank and a metering pump with ammonia or ammonium sulfate. Other nutrients, such as phosphorous, may also be added by the nutrient-supply system.

In certain embodiments, a nutrient supply system adds nitrogen, phosphorus, and/or other minerals via package feeding systems typically used in industrial wastewater treatment plants. The nutrient supply system may comprise a mix tank, a day tank, and an automated metering pump. The nutrient supply system may be used to add nutrients or minerals, such as ammonia, ammonium sulfate, and phosphoric acid.

In other non-limiting examples, the separation system comprises a membrane. In specific embodiments, the membrane may be a hollow-fiber, ultra-filtration membrane system such as a Zenon® system, or a flat-sheet submerged membrane system, such as a Kubota® system. In certain embodiments, the separation (or dewatering) system will concentrate solids from a range of 20-10,000 mg/L to a range of 1,000-50,000 mg/L, more preferably from 100-300 mg/L to 5,000-25,000 mg/L, and most preferably from 200 mg/L to 10,000 mg/L. The separation system may circulate permeate water and concentrated solids back to the reaction system, and it may be a one-stage or a multi-stage system.

In specific embodiments, the separator will receive fluid from the conduit, and it will separately return concentrated solids back to the conduit, remove concentrated solids from the system for further processing, remove filtered (solids-free) permeate water from the system, and return filtered permeate water to the conduit. This set of flows to and from the separator make it possible to control independently the solids (i.e., photosynthetic microorganisms) concentration inside the conduit, the solids concentration removed from the separator for further processing and return to the conduit, and the specific growth rate of the photosynthetic microorganisms inside the conduit.

In certain embodiments, the concentrated solids removed from the system may be shipped to a storage tank for further dewatering. A second dewatering step may concentrate the product from one percent solids to 5-50 percent solids, more preferably 10-25 percent, and most preferably 15-20 percent solids. This may be accomplished using a centrifuge, for example a decanter centrifuge (also known as a scroll or solid bowl centrifuge). Certain non-limiting examples may comprise additional filters as well. In certain embodiments, a flocculation system (for example, a polymer system) may be used to capture solids in the centrifuge, and the centrifuge may be sized so that processing of solids can be achieved during a single work shift.

Certain non-limiting embodiments comprise a processing system for converting the recovered solids into biofuels (for example, biodiesel) or other valuable products, including a semi-dry or dry “fractured” cell residual that could be a combustion fuel or have other possible uses. In certain embodiments, the product processing comprises lysis or fracturing the cells. Various methods of fracturing may be employed, including, but not limited to: thermal treatments; sonic treatments; mechanical abrasion (for example, positive displacement pumps); pressurization and sudden depressurization; abrasion and fracture aided by addition of inert media; pulsed electric field; alkali or acid treatment. In certain embodiments, additional processing methods may be performed after fracturing. For example, direct solvent or supercritical CO₂ extraction of the oil or other products from the solids may be performed. This may be followed by biodiesel production from the oil, and dewatering leftover cell fragments to approximately 10-80 percent solids, more preferably from 30-60 percent, and most preferably about 50 percent solids. In other non-limiting examples, the cells may be dried to 80 percent or more solids, more preferably 90 percent or more, and most preferably near 100 percent solids followed by solvent or supercritical CO₂ extraction of the oil for biodiesel production. In other embodiments, a product containing approximately twenty percent solids may be treated with heat, alkali and ethanol to produce a biodiesel product. Various drying methods may be used in embodiments; for example, solar drying or mechanical drying may be used to dry the product.

It is contemplated that any embodiment discussed in this specification can be implemented with respect to any method or system of the invention, and vice versa. Furthermore, systems of the invention can be used to achieve methods of the invention.

The term “conduit” or any variation thereof, when used in the claims and/or specification, includes any structure through which a fluid may be conveyed. Non-limiting examples of conduit include pipes, tubing, channels, or other enclosed structures.

The term “reservoir” or any variation thereof, when used in the claims and/or specification, includes any body structure capable of retaining fluid. Non-limiting examples of reservoirs include ponds, tanks, lakes, tubs, or other similar structures.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE FIGURES

### DESCRIPTION OF ILLUSTRATIVE EMBODIMENTS

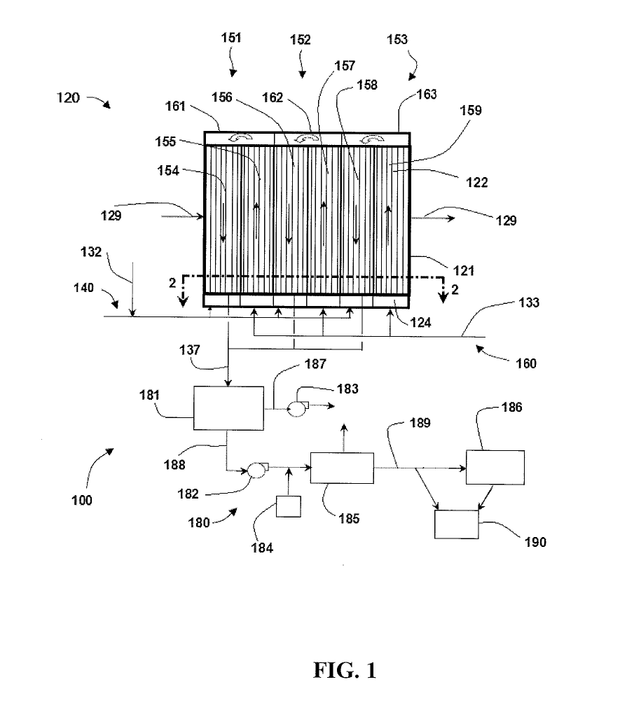

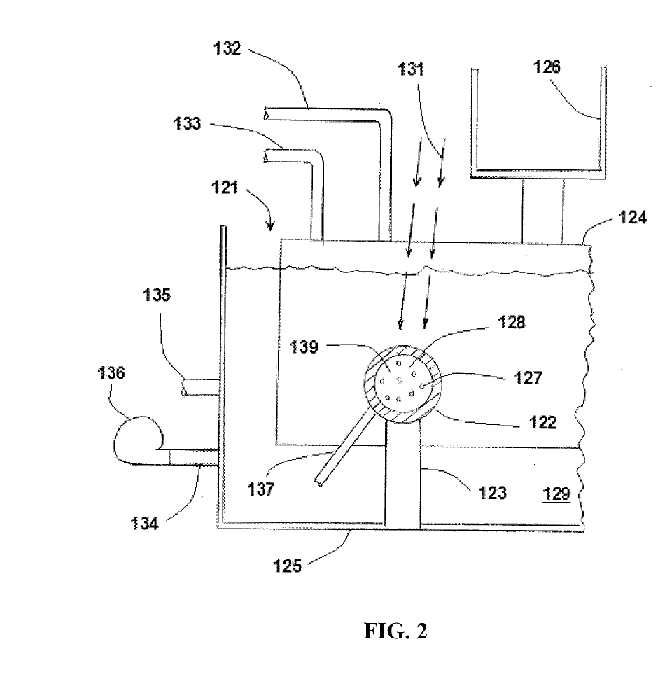

Referring initially to FIG. 1, a system 100 for growing photosynthetic cells comprises a thermal dampening system 120, a CO₂ supply system 140, a nutrient-supply system 160, and a separation system 180. Referring additionally to FIG. 2, a partial cross-sectional view of system 100 comprises an external dampening fluid 129 in a fluid reservoir 121, a conduit 122, a conduit support 123, a distribution header 124, a liner 125, and a catwalk 126. For purposes of clarification, only one conduit 122 is shown in FIG. 2. In the embodiment shown, an internal fluid 139 comprises cells 127 that grow within an inner volume 128 of conduit 122, which is comprised of a material that transmits light 131 to internal fluid 139 within inner volume 128. As shown in this embodiment, a fluid-removal pipe 137 allows internal fluid 139 and photosynthetic cells 127 to be drained or removed from inner volume 128. Also shown in FIG. 2, a CO₂ pipe 132 and a nutrient-supply pipe 133 are coupled to distribution header 124. The embodiment shown in FIG. 2 comprises a fluid inlet pipe 134 supplying external fluid 129 to reservoir 121 and a fluid outlet pipe 135 allowing external fluid 129 to exit reservoir 121.

In the embodiment shown, a pump 136 circulates external fluid 129 through reservoir 121 via inlet pipe 134 and outlet pipe 135. In certain embodiments, thermal dampening system 120 comprises fluid reservoir 121 and external fluid 129. In other embodiments thermal dampening system 120 also comprises pump 136, inlet pipe 134 and outlet pipe 135 and other associated control equipment, such as temperature and flow control devices.

Reservoir 121 may be divided into flow segments 151, 152, and 153. Each flow segment 151-153 may be further divided into opposing flow sections 154-159 and end sections 161-163. For example, internal fluid 139 may flow from distribution header 124 through flow section 155, end section 161 and back through flow section 154 to distribution header 124. In one embodiment, air lift pumps (not shown) proximal to (or integral with) distribution header 124 provide motive force to circulate internal fluid 139. A pump may provided at the inlet end of each flow section 154-159, or each segment 151-153 may use a single pump to circulate flow of internal fluid 139 within the segment. In certain embodiments, a gas containing CO₂ may be injected into the air driving the air flow pumps. In other embodiments, a gas containing CO₂ may be injected directly into distribution header 124 or into the nutrient-supply pipe 133.

In certain embodiments, CO₂ supply system 140 may comprise a pipe 132 that supplies a gas comprising CO₂ to inner volume 128 of conduit 122. In a non-limiting example, pipe 132 may be coupled to a combustion off-gas. In a specific non-limiting embodiment, pipe 132 may be coupled to a flue gas vent from a power plant. CO₂ supply system 140 may also comprise equipment associated with pipe 132; for example, CO₂ supply system 140 may comprise equipment used to regulate the flow of CO₂ and/or remove unwanted substances from the CO₂ supply stream.

In certain embodiments, the nutrient-supply system 160 may comprise a nutrient-supply pipe 133 that supplies nutrients and minerals to inner volume 128 of conduit 122. In a non-limiting example, pipe 133 may transmit either a nitrogen gas or ground water containing nitrates to inner volume 128. Nutrient-supply system 160 may also comprise equipment associated with pipe 133; for example, nitrogen supply system 160 may comprise equipment used to regulate the flow of nutrients and/or remove unwanted substances from the nitrogen supply stream.

Separation system 180 comprises equipment used to separate photosynthetic cells 127 from internal fluid 139. In the specific embodiment shown in FIGS. 1 and 2, separation system 180 comprises liquid-removal pipe 137, a membrane separator 181, and a recycle pump 183. Separation system 180 may be followed by a feed pump 182, a polymer injector 184, a centrifuge 185, and a dryer 186.

During operation of system 100, cells 127 are grown in inner volume 128 through photosynthesis. CO₂ pipe 132 supplies CO₂ to distribution header 124 or upstream of the distribution header. The nutrient-supply pipe 133 supplies nutrients and minerals to distribution header 124, which is coupled to inner volume 128 of conduit 122. At least a portion of conduit 122 is submerged in external fluid 129, which dampens thermal fluctuations or variations of inner volume 128. The temperature of external fluid 129 can be maintained by a temperature control mechanism such as a heat exchanger (not shown in FIG. 1 or 2) or the cooling system for a combustion power plant. In certain embodiments, external fluid 129 is maintained at a desired temperature and/or has a higher specific heat than atmospheric air. External fluid 129 may reduce thermal fluctuations of inner volume 128 caused by factors such as changes in outside temperatures due to natural weather patterns or day-to-night variations. In certain embodiments, reducing thermal variations may promote growth of cells 127, which is accomplished via a reaction of light 131 and the CO₂ and nitrogen supplied from pipes 132 and 133, respectively.

As shown in the embodiment of FIG. 1, internal fluid 139 (comprising cells 127) can be removed from inner volume 128 via liquid-removal pipe 137 which is coupled to membrane separator 181. In certain embodiments, separator 181 is a Zenon-type membrane that removes cells 127 from external fluid 139. In the embodiment shown in FIG. 1, a portion 187 of fluid 139 is recycled back to distribution header 124 via recycle pump 183, and a solids-containing portion 188 is fed to centrifuge 185 via feed pump 182.

In the embodiment shown, polymer injector 184 injects polymer into solids-containing portion 188 before it reaches centrifuge 185. In certain embodiments, a product stream 189 exiting centrifuge 185 comprises 15-20% solids. In the embodiment shown in FIG. 1, a portion of product stream 189 can be fed to dryer 186. Product stream 189 may be converted to a biomass 190 and then biofuel or biodiesel through techniques such as lysis or hexane extraction.

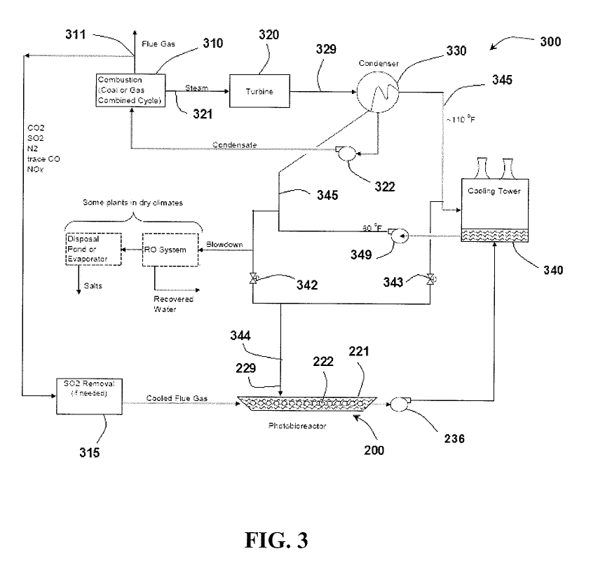

Referring now to FIG. 3, an embodiment is shown comprising a cell-growing system 200 integrated with a power plant 300. System 200 comprises a fluid reservoir 221 and a series of conduit 222 similar to that of system 100 shown in FIGS. 1 and 2. In the embodiment of FIG. 3, CO₂ and thermal dampening fluid are provided by existing systems commonly found in power plants. Power plant 300 comprises a turbine 320 that is powered by a steam supply 321 provided by a boiler 310. In certain embodiments, exhaust steam 329 from turbine 320 is condensed by a condenser 330 and recycled back to boiler 310 via a recycle pump 322. In the embodiment shown, boiler 310 produces a combustion off or flue gas 311 (containing CO₂) that is sent to system 200 and used in the production of cells. In the embodiment of FIG. 3, a scrubber 315 can be used to remove certain gases, including SO₂, from flue gas 311.

In the embodiment shown in FIG. 3, power plant 300 comprises a cooling tower 340 that supplies cooling water 345 to condenser 330. Cooling water 345 exits cooling tower 340 at a certain temperature (approximately 80 degrees F. in the embodiment shown) and passes through condenser 330, where the temperature is increased to a higher temperature (approximately 110 degrees F. in the embodiment shown) before returning to cooling tower 340. Cooling tower 340 then cools cooling water 345 down to a lower temperature and a cooling tower pump 349 circulates cooling water 345 through the condenser 330.

In certain embodiments, a first control valve 341 is coupled to the cooling water exit (where cooling water 345 is at a lower temperature) and a second control valve 342 is coupled to the cooling water return (where cooling water 345 is at a higher temperature). Control valves 341 and 342 may also be coupled to a supply line 344 that supplies an external fluid 229 (in this example, a blend of cooling water 345 from the condenser supply and return lines) to system 200. A control system (not shown) can be used to control the temperature of external fluid 229 by opening or closing control valves 341 and 342. The temperature of external fluid 229 can be controlled to any temperature between the cooling water exit temperature (in the example shown, 80 degrees F.) and the cooling water return temperature (110 degrees F. in the example shown). For example, if valve 342 were open and valve 343 were fully closed, the temperature of external fluid 229 would be the temperature of the cooling water exit. If valve 343 were open and valve 342 fully closed, the temperature of external fluid 229 would be the temperature of the cooling water return. If both valves 342 and 343 are partially open, the temperature of external fluid 229 would be somewhere between the cooling water exit and return temperatures. In the embodiment shown in FIG. 3, external fluid 229 can be circulated through fluid reservoir 221 so that it contacts conduit 222 and reduces thermal variations of conduit 222. A pump 236 pumps external fluid 229 back to cooling tower 340.

Integrating system 200 with existing equipment and systems at power plant 300 allows for higher efficiency of system 200. For example, flue gas or combustion off gas 311 may provide an existing source of CO₂ that requires minimal expenditures of capital or energy to recover. In addition, power plant 300 may provide a source for cooling water 345 that can be used as a fluid for dampening thermal variations in conduit 222. Again, this system can be incorporated with minimal expenditures. Although integration with a power plant may increase efficiency of operation, it is understood that other embodiments do not utilize such integration.

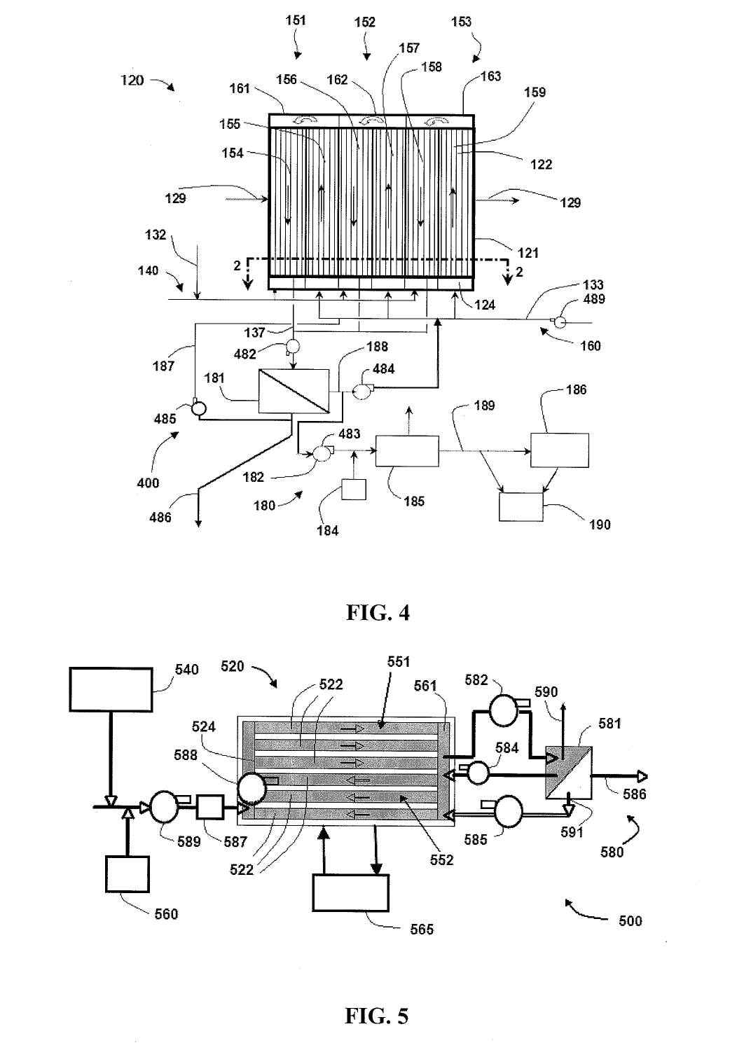

Referring now to FIG. 4, an alternative embodiment of a system 400 for growing photosynthetic cells comprises similar features to the previously-described embodiment, with certain revisions to the process and equipment. Elements that are equivalent to those in the previously-described embodiment are given equivalent reference numbers.

Elements of system 400 that are equivalent to elements of system 100 are given equivalent reference numbers. However, in the embodiment shown, system 400 comprises a different piping and pumping arrangement as compared to system 100. For example, system 400 may comprise a nutrient feed pump 489 that can be used to provide flow within nutrient-supply system 160. System 400 may also comprise additional pumps in fluid communication with membrane separator 181. For example, system 400 may comprise a separation feed pump 482 that pumps internal fluid 139 from the internal volume of a conduit (e.g., internal volume 128 of conduit 122) to separation system 180.

In the embodiment shown, system 400 may also comprise a solids feed pump 483 that can be used to feed solids separated from membrane separator 481 to centrifuge 185 (or associated processing equipment). System 400 may also comprise a solids return pump 484 that can be used to recycle solids from membrane separator 481 back to nutrient-supply line 133. In addition, the embodiment shown comprises a filtered permeate recycle pump 485 that can pump filtered fluid back to the internal volume of a conduit (e.g., internal volume 128 of conduit 122). System 400 can also comprise a permeate or liquid drain 486.

System 400 as shown comprises flow segments 151, 152, and 153 (with opposing flow sections 154-159 and end sections 161-163), similar to system 100. It is understood that other embodiments may have fewer or more flow segments. In certain embodiments, system 400 may only have one flow segment. It is also understood that system 400 may comprise any CO₂ injection location in fluid communication with the internal volume of conduit or other location of photosynthesis.

Referring now to FIG. 5, a schematic diagram illustrates an embodiment of a system 500 for growing photosynthetic cells that is similar to previously-described embodiments. Unless stated otherwise, elements of system 500 are equivalent to similarly named and similarly numbered elements in previously described embodiments. In this schematic, system 500 comprises opposing flow sections 551 and 552 and coupling portions 524 and 561 (which enable flow section 551 to be in fluid communication with flow section 552). Flow sections 551 and 552 comprise enclosures or conduit 522, in which the previously-described photosynthesis takes place. It is understood that the term “conduit” as used herein is to be construed broadly and includes any container capable of holding fluid. In this exemplary embodiment, system 500 comprises a thermal dampening system 520, a CO₂ supply system 540, a nutrient supply system 560, and a temperature control system 565. System 500 also comprises a separation system 580 comprising a clarifier or membrane separator 581, which separates the concentrated-solids retentate or harvested biomass 590 from the effluent 591.

In the exemplary embodiment shown, system 500 comprises a nutrient feed pump 589 that can be used to provide flow within nutrient-supply system 560, as well as a separation feed pump 582 that feeds biomass material to separation system 580. System 500 may also comprise a sterilization system 587 that can be used to sterilize nutrients before they enter conduit 522 and an internal recirculation pump 588 used to circulate fluid in flow sections 551 and 552. In addition, system 500 may comprise a solids return pump 584 that can be used to recycle solids from membrane separator 581 back to conduit 522. In the embodiment shown, system 500 may comprise a filtered permeate recycle pump 585 that can pump filtered fluid back to the internal volume of a conduit 522. System 500 can also comprise a permeate or liquid drain 586.

### Example

In a specific non-limiting example, a system for growing cells comprises a pond that is 130 meters square and 5 feet deep for use as a thermal-dampening system. The pond is formed with earthen embankments in a generally level area and has a synthetic membrane liner. About 540 parallel 100-meter long, clear 6-inch diameter PVC pipes extend across the pond. The pipes are submerged about 3 to 4 feet below the surface. The pipes are supported from the bottom of the pond by pipe stands, and each end of the pipes is in fluid communication with a header.

The pond is divided into three segments, with each segment divided into two counter-flowing sections. Internal flow of fluid in the pipe flows from one distribution header and across the pond through one section of pipe. The fluid then enters a second header, where it is directed towards a second section of pipe that flows counter to the first section of pipe. After exiting the second section of pipe, the fluid re-enters the first header and continues the cycle. Because each segment is independent of the other segments, different operating conditions can be maintained within each segment of the pond (if desired). For example, one segment of the pond may be shaded, while the other segments are not shaded. In addition, different flow rates or nutrient levels may be maintained in different segments to determine optimum operating conditions.

The motive force for the internal fluid flow is provided by a series of air lift pumps incorporated in the first header. In this example, there are 12 pumps (four in each segment) that provide 2,500 L/min of flow at one meter of dynamic head. The pumps are connected to an air blower that provides approximately 1,500 cubic meters/hour of air flow. The air from the air blower is injected with CO₂ gas obtained from a flue gas at an adjacent plant or other production facility.

In addition, a nutrient and mineral supply system is used to add nutrients to the internal fluid of the pipes via one of the headers. This system is a package system that is typically found in industrial wastewater treatment plants. The system includes mix tanks, storage tanks and automated metering pumps to add nutrients such as ammonia, ammonium sulfate, and phosphoric acid to the internal fluid. The level of nutrients can be controlled independently for each segment.

External flow of fluid outside of the pipe is provided by a pond circulation system. This system can be incorporated into a cooling water supply system of the existing plant to provide a controlled-temperature fluid for the pond. The plant cooling water acts to dampen any temperature fluctuations resulting from changes in atmospheric conditions. Cooling water from the plant is pumped into the pond and flows transversely across the rows of tubes. The cooling water is then pumped from the pond back to the plant so that the temperature may be reduced by the cooling tower. The temperature of the pond water can be maintained at any temperature between the temperature of the cooling water exiting the plant cooling tower (typically about 80 degrees F.) and the temperature of the cooling water returning to the plant cooling tower from other plant equipment (typically about 110 degrees F.).

Catwalks are placed above the pond level that allow personnel to access various areas of the system. The catwalks run longitudinally and transversely across the pipes, allowing maintenance activities, such as cleaning of the pipes, to be performed.

During operation, cyanobacteria cells (in accordance with U.S. Provisional Patent Application Ser. No. 60/853,285, entitled “Modified Cyanobacteria”, filed on or about Oct. 20, 2006 by Willem F. J. Vermaas) are cultured in the internal fluid within the pipe. The clear PVC pipe allows natural light to pass through the wall of the pipe and exposes the internal fluid to the natural light. In addition, the clarity of the cooling water in the pond is also maintained to allow natural light to pass through the pond. The natural light, CO₂, fixed nitrogen, and other nutrients existing within internal fluid of the pipes provide the needed elements for photosynthesis to occur, as explained more fully in the above-referenced U.S. Provisional Patent Application filed on or about Oct. 20, 2006 by Vermaas entitled “Modified Cyanobacteria.” In addition, the external fluid can be used to reduce thermal fluctuations and maintain an optimum temperature range for the growth of the cyanobacteria. As a result, cyanobacterial cells are efficiently cultured within the pipe.

Within each segment, there are liquid removal pipes that allow internal fluid to be drained from the pipes and/or headers. The internal fluid is initially passed through a Zenon® or Kubota® membrane that increases solid concentration from about 200 mg/L to about 1 percent solids. Pumps and pipes are provided to remove filtered permeate from the system, return filtered permeate to the photobioreactor, remove concentrated solid for further dewatering and product recovery, and recycle concentrated solids back to the photobioreactor.

A flocculation system is used to inject polymer into the harvested solids flow, which can then be sent to a storage tank (if necessary) or solid bowl centrifuge, where the solid concentration is increased to about 15-20 percent solids.

The solids can then be sent to a dryer (if needed) and converted to a biomass. The dewatered biomass may then be processed through lysis or fracturing the cells via thermal or sonic treatments; mechanical abrasion; pressurization and depressurization; abrasion and fracture by addition of inert media; pulsed electric field; or alkali or acid treatment.

After fracturing, additional processing may include direct solvent or supercritical CO₂ extraction of the oil from the solids, followed by biodiesel production from the oil. In addition, the leftover cell fragments can be dewatered to approximately 50 percent solids. The desired product, such as oils for biofuel, can then be extracted. As an alternative, the cells may be dried to near 100 percent solids, followed by solvent or supercritical CO₂ extraction of the oil for biodiesel production. Still other processing methods include treating the 20 percent solid product with heat, alkali and ethanol to produce a biodiesel product directly. Drying the product may be accomplished via mechanical equipment of solar drying.

A source of make-up water may be needed to replace the internal process fluid lost during production of the cyanobacteria in the pipe. The filtered water removed from the system may be discharged to a receiving water, to a wastewater treatment facility, or to another beneficial use. After the pipes are initially filled, the amount of make-up fluid required will be minimal because most of the water is recovered and recycled within the system. The system does include a small wastewater blowdown to control inorganic or organic impurity build-up in the process water. The wastewater could be sent to a treatment plant offsite.

It may be necessary to periodically completely drain the internal process fluid from the pipes to perform maintenance on the system. A second, smaller pond that is lined and lower in elevation than the primary pond may be used to receive the internal process fluid before it is sent for treatment.

Operating parameters of the system can be controlled by Programmable Logic Controllers (PLCs) that would allow the system to run automated for periods of time. The PLCs can be used to log data, as well as transmit operating conditions to off-site personnel. It is recommended that on-site personnel be present during daylight hours, and that the system run automated overnight.

All of the systems and/or methods disclosed and claimed in this specification can be made and executed without undue experimentation in light of the present disclosure. While the systems and methods of this invention have been described in terms of preferred embodiments, it will be apparent to those of skill in the art that variations may be applied to the systems and/or methods and in the steps or in the sequence of steps of the method described herein without departing from the concept, spirit and scope of the invention. More specifically, it will be apparent that other types of equipment may be substituted for the specific equipment types described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Publication No. 2002/0072109

U.S. Pat. No. 4,242,455

U.S. Pat. No. 4,350,765

U.S. Pat. No. 4,413,058

PCT Application No. WO/88/09379

U.S. Publication No. 2002/0042111

U.S. Provisional Patent Application Ser. No. 60/853,285, entitled “Modified Cyanobacteria”, filed on or about Oct. 20, 2006 by Willem F. J. Vermaas

PCT Application No. ______, entitled “Modified Cyanobacteria”, filed on or about Oct. 20, 2007 by Willem F. J. Vermaas.

1. A system for growing photosynthetic cells comprising:
at least one conduit comprising an outer surface, an inner surface, an inner volume, a length, and at least a portion that permits sunlight to pass into the inner volume during use, wherein at least a portion of the at least one conduit is exposed to sunlight during day;
a thermal dampening system in operable relationship to the at least one conduit;
a CO₂ supply system configured to supply CO₂ to the inner volume during use;
a nutrient-supply system configured to supply nutrients to the inner volume during use; and
a separation system configured to remove the photosynthetic cells from the at least one conduit during use.

2. The system of claim 1 further comprising:
a fluid control system configured to:
remove a substantially solids-free permeate from the conduit;
recycle a portion of the substantially solids-free permeate back to the conduit;
remove a concentrated-solids retentate from the system; and
recycle a portion of the substantially solids-free permeate back to the conduit.

3. The system of claim 1, wherein the nutrients comprise a component of nitrate or another fixed nitrogen compound.

4. The system of claim 1, wherein the photosynthetic cells are further defined as cyanobacteria.

5. The system of claim 1, further comprising a mineral supply system configured to supply minerals to the inner volume during use.

6. The system of claim 1, wherein the portion that permits sunlight to pass into the inner volume during use is a clear portion comprised of glass, clear polyvinyl chloride, or another polymer.

7. (canceled)

8. The system of claim 1, wherein the at least one conduit comprises a tube with a circular cross-section.

9. The system of claim 7, wherein the tube is approximately four to ten inches in diameter.

10. The system of claim 1, wherein the at least one conduit is at least one hundred feet long.

11. The system of claim 1, wherein the thermal dampening system is configured to circulate a fluid in contact with the at least one conduit during use.

12. The system of claim 1, wherein the thermal dampening system comprises a fluid reservoir in which at least a portion of the at least one conduit is submerged in the fluid reservoir.

13.-15. (canceled)

16. The system of claim 1, wherein the CO₂ supply system is configured to inject flue gas into a liquid in fluid communication with the inner volume during use.

17. (canceled)

18. The system of claim 1, further comprising a pump configured to circulate a fluid within the conduit.

19.-21. (canceled)

22. The system of claim 1, further comprising a distribution header in operable relationship with the at least one conduit.

23. The system of claim 22, wherein the distribution header is configured to receive CO₂ injection during use.

24. (canceled)

25. The system of claim 1, wherein the nutrient-supply system is configured to supply ground water to the inner volume during use.

26. The system of claim 1, wherein nutrient-supply system comprises a storage tank and a metering pump.

27. The system of claim 1, wherein the nutrient-supply system comprises ammonia or ammonium sulfate.

28. The system of claim 1, wherein the separation system comprises a membrane separator.

29. The system of claim 28, wherein the membrane separator comprises a first outlet configured to remove a concentrated-solid retentate from the system, a second outlet configured to remove a substantially solid-free permeate from the system, a third outlet configured to recycle a concentrated-solid retentate to the conduit, and a fourth outlet configured to recycle a substantially solid-free permeate to the conduit.

30. A method of growing photosynthetic cells comprising:
culturing photosynthetic cells in an inner volume of one or more conduits;
supplying CO₂ and nutrients to the inner volume;
exposing the CO₂ and nutrients to natural light;
dampening thermal variations in the conduits; and
removing cells from the inner volume.

31. The method of claim 30, wherein the cells are further defined as cyanobacteria.

32. The method of claim 30, wherein the nutrients comprise fixed nitrogen.

33. The method of claim 30, wherein dampening thermal variations comprises contacting an outer surface of the conduits with a fluid.

34. The method of claim 33, wherein the temperature of the fluid is controlled.

35. The method of claim 30, wherein the CO₂ is supplied by a flue gas.

36. The method of claim 30, wherein the nutrients comprise fixed nitrogen supplied by a fluid selected from the group consisting of: ground water, ammonia, and ammonium nitrate.

37.-38. (canceled)

39. The method of claim 30, wherein the photosynthetic cells are removed by a membrane.

40. The method of claim 30, wherein the conduits are submerged in a fluid reservoir.

41. (canceled)

42. The method of claim 30, wherein the CO₂ in the inner volume is maintained at about 0.03% to 5%.

43. (canceled)

44. The method of claim 30, wherein the nutrients in the inner volume comprise fixed nitrogen maintained at about 0.5-10 mM.

45. (canceled)

46. The method of claim 30, wherein the temperature in the inner volume is maintained at about 10-60 degrees Celsius.
